(19) United States
(12) Patent Application Publication
(10) Pub. No.: US 2001/0025139 A1
(43) Pub. Date: Sep. 27, 2001

(54) Multivariate cardiac monitor

(76) Inventor: Justin D. Pearlman, Brookline, MA (US)

Correspondence Address: PERKINS, SMITH & COHEN LLP, ONE BEACON STREET, 30TH FLOOR, BOSTON, MA, 02108, US

(21) Appl. No.: 09/773,167

(22) Filed: Jan. 31, 2001

Related U.S. Application Data
(60) Provisional application No. 60/179,192, filed on Jan. 31, 2000.

(51) Int. Cl. A61B 10/00

(52) U.S. Cl. 600/301

(57) ABSTRACT

Multiple electrode contacts make electrical connections to the anterior and/or posterior chest for multi-variate characterization of the electrical activation of the heart. A central processing unit derives synthetic composite electrographic signals as well as flag signals for specific purposes. A preferred embodiment uses this system to trigger or gate magnetic resonance imaging, eliminating or reducing problems from small or inverted R-waves, lead detachment, noise, flow signal, gradient changes, and rhythm changes, more reliably flagging the onset of electrical activation of the ventricles. Additional derived data are ST-segment shifts, filling times, and respiratory cycle. Filling times may be used for greatly improved imaging in the presence of rhythm disturbances, such as atrial fibrillation. Respiratory cycle may be used as a respiratory trigger to control for the effects of breathing on the heart position and image quality.

### CROSS REFERENCE TO RELATED APPLICATIONS

This application claims priority of U.S. Provisional Application Ser. No. 60/179,192 entitled MULTIVARIATE CARDIAC MONITOR FILED Jan. 31, 2000 by the present inventor and which is also incorporated herein by reference.

### FIELD OF THE INVENTION

This invention relates generally to medical diagnostic testing and in particular, to a multivariate sensor system based on an electrode assembly.

### BACKGROUND OF THE INVENTION

Electrocardiographic (ECG) measuring systems generally apply 3 electrodes (to the chest or 10 electrodes (4 limbs and 6 specific points on the chest) to the skin, and, through a differential operational amplifier (OP-AMP), report signal differences between a selected pair of electric contacts or electrodes or between an electrode and a summed reference. The electrical activity thus monitored is generated by a sequence of ion movements in the heart that depolarize (release) and then repolarize (rebuild) an ionic charge distribution across cell membranes, that relates to actuation of contraction of the heart muscle. By convention accepted in the art (with reference to the figures), a “12 lead” ECG consists of lead pairings I, II, III, avR, avL, avF, v1, v2, v3, v4, v5, and v6, where lead I reports the voltage difference between an electrode on the left arm and another on the right arm; lead II left arm vs. foot; lead III right arm vs. foot; Lead aVR reports right arm vs. combined reference of left arm and foot; aVL left arm vs. right arm and foot; aVF foot vs. left arm and right arm; and the v-leads (v1-v6, v for voltage) represent a series of prescribed positions across the front of the chest vs. the combined reference of left arm, right arm and feet. The American Heart Association and the Cardiac Society of Great Britain defined the standard positions and the wiring for the chest leads v1-v6 in 1938 (Barnes AR, Pardee HEB, White PD. et al. Standardization of precordial leads. Am Heart J 1938;15:235-239). Emanuel Goldberger added the augmented limb leads aVR, aVL and aVF to Einthoven's three limb leads and the six chest leads in 1942, constituting the 12-lead electrocardiogram that is used today. ECG systems are widely used for diagnosis of rhythm changes, metabolic effects, and heart damage.

The ECG signal is commonly described in terms of a sequence of waves called P wave, QRS complex, and the T-wave (originally described by Willem Einthoven, Einthoven W. Ueber die Form des menschlichen Electrocardiograms. Arch f d Ges Physiol 1895;60:101-123; Nobel prize awarded 1924). The QRS complex may consist of just R wave or RS or qR or qS, where q, if present, is an initial down-going voltage deflection, R, if present, is the first up-going deflection deflection after the p-wave, and S, if present, is a subsequent down-going deflection (if there are further up-going and down-going waves in the QRS, those are labeled R′, S′, then R″, S″, respectively). The P-wave corresponds to electric activation of the small chambers of the heart. The R-wave or QRS complex corresponds to electrical activation of the large chambers of the heart. The T wave corresponds to the staggered end of electric charge redistribution recovery from the electrical activation of the large chambers.

Alternatively, the heart has been modeled for simplicity as a 3D electric dipole represented by orthogonal ECG tracings, and xy, xz and yz loop plots known as vectorcardiograms (VCG's), but VCG's are not relied on and are unpopular clinically for diagnostics or monitoring (E. Frank: The Image Surface of a Homogenous Torso, Am. Heart J. 47:757, 1954). Vectorcardiograms are based on 3 orthogonal voltage loop plots representing an electric dipole that changes length and orientation cyclically. The heart is not that simple, so the model introduces error well described in the literature. The vector model is not as powerful at separating unwanted signals as is the multivariate method of this invention, it does not provide ST segment monitoring, and it requires a more difficult set up to be done properly. The underlying model has an estimated 10% error, because the heart is not simply a 3D electric dipole, different lead positions have distinct local information, and more than 4 leads are needed to reproduce the ECG (G. E. Dower, H. B. Machado, J. A. Osbone: On Deriving the Electrocardiogram from Vectorcardiographic Leads, Clin. Cardiol. 3:87, 1980; L. Edenbrandt, O. Pahlm: Vectorcardiogram Synthesized from a 12-lead ECG: Superiority of the Inverse Dower Matrix, J Electrocardiol 21:361, 1988).

ECG's are used to detect the “R-wave” (the initial up-going component of the QRS). R-wave detection is used to synchronize imaging systems with the position of the beating heart, e.g., for triggering data collection (a strobe-like method to collect data at specific times to effectively freeze the motion of the heart), or for gating the data (to sort collected data in relation to the timing of activation of the heartbeats).

Following the R-wave and before the T wave there is an early electric recovery period reflected by a voltage called the “ST segment.” In certain lead pairings, the ST segment may be depressed or elevated with respect to the baseline of the ECG signal, and in particular with respect to the extrapolation of the P-R segment. It may become depressed when blood supply to the heart is insufficient for the normal metabolism (ischemia), or elevated when there is new or recent damage to the heart muscle (injury current), or vice versa if ischemia or damage is visible from the opposite side of the heart.

Such ST segment deviation is typically evident only in particular lead pairings, which may or may not include standard leads. For example, infarctions on the posterior or right aspects of the heart may be missed in a 12-lead ECG, and an enlarged or unusually positioned heart may not be adequately assessed with the standard 12-lead system.

When such circumstances are suspected, clinical practice calls for additional lead placements, e.g., V7, V8, V9, V4R, and V5R.

A recent study showed that continuous monitoring of the ST segment following a heart attack provides a good predictor of the amount of damage. In particular, the intensity and duration of myocardial ischemia (both reflected by the estimated areas under the ST-trend curve) determine the extent of myocardial damage infarct size and ejection fraction in patients with acute myocardial infarction who receive clot-busting therapy (Karel G.M. Moons PhD, Peter Klootwijk MD PhD, Simon H. Meij MSc, Gerrit-Anne van Es PhD, Taco Baardman MD, Timo Lenderink MD, Marcel van den Brand MD PhD, J. Dik F. Habbema PhD, Diederick E. Grobbee MD PhD, Maarten L. Simoons MD PhD. Continuous ST-Segment Monitoring Associated With Infarct Size and Left Ventricular Function in the GUSTO-I Trial, Am Heart J 138(3):525-532, 1999). Also in association with ischemia or injury, the T-wave may change form or invert.

Prior art solutions to problems encountered during electrocardiography include using light emitting diodes to flag poor electrode contact because electrodes may become detached during data collection. One device uses a microprocessor to trigger an alarm when a drop in impedance below a threshold value is detected, simultaneously activating an automatic search for alternative lead combinations that may be intact. Another device applies additional leads to use as alternates depending on patient size, embedding the leads in a uniformly weighted pad. Another prior art device enables amateur application of multiple leads in the general region of the heart for computer selection of a lead that appears to have correct position.

Despite the application of multiple leads, these prior instruments and methods merely provide alternates for selection of a preferred electrode set to use in the conventional manner of reporting signal differences between a pair of voltage sources and/or require particular lead placements. They assume that there is a best subset of standard combinations and standard positions to use for gathering a usual ECG signal. In normal healthy subjects, with standard lead placements, that may be true; but diseased patients generally have changes in the heart resulting in changes in the ECG signal from standard lead pairs. In particular, myocardial infarction, or heart attack, typically results in loss of R-wave height.

Triggering and gating are impaired if the R-wave is not the expected tallest narrow spike in the ECG. Taller R-waves may be found if observed from other, non-standard, electrode locations. To address the problem of failed ECG triggering, filters have been applied ECG signal to reduce signal at frequencies not of interest; that can help but does not reliably resolve the problem.

Even with a normal ECG, there are “electrically silent areas” of the heart in which ischemia or injury may occur without the usual evidence of ischemia or injury in the standard lead position ECG, as mentioned above. Patients with enlarged or repositioned hearts may be better evaluated from non-standard lead positions. As a subject breathes in and out there is a “baseline artifact” which may interfere with standard interpretation of the signals, but which may prove useful in reporting the phase of breathing. Also, there is a variation in the interval from one heart beat to the next (“R-R interval”), allowing increased or decreased filling of the chambers, and thus changes in the size of the heart that may impair the goal of ECG triggering or ECG gating. In response to changes in filling, the heart changes its contractility (strength and rate of contraction) for the subsequent cycle(s). Also, incorrect placement of the chest leads v1-v6 can produce false indications of ischemia or infarction.

Magnetic Resonance Imaging (MRI) is an example of an imaging device that uses the height of the R-wave as a trigger to synchronize data collection to the heartbeat activation and effectively freeze the motion of the heart. Until recently, MRI took over 20 minutes to build one or more images of the heart as a composite from multiple heartbeats. New MRI systems can acquire images in less than 20 seconds, with some methods completing an image in less than half a second. With such capabilities, it is now possible to follow changes in the heart from beat to beat. For example, one may observe the arrival of a blood-born contrast agent and determine if there are areas of impaired blood delivery. Such methods need, more than ever, a reliable detection of the electrical activation of the large chambers of the heart. Newer MRI systems also have higher magnetic fields than in the past, resulting in greater induction of an electrical signal due to the pulses of blood moving along in the great vessels. That signal generally adds to the normally lower “T-wave.”Consequently, the R-wave is often not the tallest wave. Also, MRI applies controlled magnetic fields to encode the data it collects for imaging. The newer faster imaging methods use improved hardware to change the magnetic field more quickly, inducing higher, narrower, electric signals that commonly obscure the R-wave. Baseline artifact related to the respiratory cycle may be exaggerated.

It remains desirable to perform accurately medical diagnostic testing on the heart, in the presence of disturbing signals, or with imperfect lead placements, such as in settings where time or expertise are limited. Likewise it remains desirable to obtain diagnostic signals when signal character is non-standard due to disease, or when the signal changes after the subject is advanced into an imaging system. Also, it is desirable to extract information about the respiratory cycle.

It is therefore an object of this invention to provide a means for rapid placement of electrical contacts.

It is also an object of this invention to provide a means for collecting and analyzing data from multiple contacts in order to characterize the electrical activation.

It is an object of this invention to generate one or more signals useful for diagnostics, triggering, or gating.

It is a further object of this invention to assess respiration.

It is still a further object of this invention to provide a simple and rapid means of forming multiple electrical contacts for diagnostic monitoring.

It is another object of the present invention to reliably represent the electrical activation of the large chambers of the heart, especially the narrow, tallest peak used for triggering imaging systems.

It is another object of the present invention to provide a method and apparatus to identify ischemia or injury to the heart.

It is an object of the present invention to report the results in a synthetic signal.

It is yet another object of the present invention to provide a method and apparatus to compare signals inside and outside the imaging system.

It is another object of the present invention to provide a method and apparatus to identify the electrical activation of the small chambers of the heart.

It is still yet another object of the present invention to provide a method and apparatus to identify and flag aberrant heartbeats.

It is yet a further object of the present invention to provide a method and apparatus to analyze cycle lengths to enable triggering based on comparable filling periods, particularly for cases of rhythm disturbances, such as atrial fibrillation, which currently have been considered relative contraindications to gated imaging.

It is another object of the present invention to provide a method and apparatus to analyze the “respiratory artifact,” and to identify the phase of breathing, and to provide a corresponding signal for control.

### SUMMARY OF THE INVENTION

The problems of performing diagnostic testing on the heart are solved by the present invention of a new electrode-based monitoring system that uses multiple electrodes to create a multivariate characterization of the status of the heart (or other organ). An example of multivariate characterization is the description of a person in terms of height, weight, sex, eye color, interests, culture, education, and so on. The present invention collects multivariate data from contacts distributed on the body, and derives from the multivariate data a synthetic or composite signal for specific purposes. A synthetic or composite signal refers to a signal that is computed or derived from measured data, but may be different in form. A synthetic ECG is a signal that represents and looks like a standard ECG but is computed or derived from data that may be non-standard.

The present invention analyzes data from multiple leads to generate a multivariate characterization of the events of interest. In the preferred embodiment, wire from an electrode is paired and twisted with wire from the same location but not making electrical contact with the chest. The wire is resistive to reduce induction of stray signals, e.g., a 24 inch carbonized wire with 200,000 ohms resistance (impedance) end to end.

A plurality of such lead pairs is applied to the anterior and/or posterior and/or side(s) of the chest wall as an array, harness, vest, partial vest or shoulder holster. These leads go to a battery-powered magnetic field-compatible processing unit. Lead pairs go to a differential operational amplifier, preferably an instrumentation amplifier, to eliminate stray signal common to both (instrumentation amplifiers provide 100 dB common mode rejection). Alternatively, leads may be used that are not physically paired to a matched location; instead, pairings with one or more common references may be used for common mode rejection. Optionally a second level of common mode rejection may be applied to the resultant signals from electronically paired leads.

All processing may be completed in a first processing unit, which may be battery-powered, or the signals may be multiplexed and converted to optical or other forms of signal for transmission to a second processing unit. The linkage between such processing units is characterized by a transmit end and a receive end. The conversion of signal for transmission may utilize an analog to digital converter (ADC), which may be a stand alone component or integrated with a microprocessor. The optical cable linkage may be plastic, e.g., passing 890 nanometer short wavelength light to support up to 125 megabits/second data transmission. The receive end may use an integrated circuit transmitter assembly to convert the data stream to a form useful for analysis, optionally with sigma-delta modulation, for a target bandwidth of 0.01-200 Hertz (low frequency near 0.3 Hertz reports respiratory effects; high frequencies contribute to signal fidelity but also noise; optionally the circuit will include pre-charging to increase the low frequency response time at start-up).

The processing compares multivariate signals to a model and/or training data to identify desired features of the signal. Training data may be any combination of historic, empiric, model, or actual data from others or from the subject to be observed. Desired features may include electrical activation of the smallest chambers (P-wave timing), electrical activation of the large chambers (R-wave timing, QRS form), baseline deviation of early repolarization (ST-segment shifts), staggered repolarization (T-wave form), respiratory cycle from baseline artifact, temporal averages and beat-to-beat variations.

The multivariate data may be processed first to reduce or eliminate bad data lines, artifacts, and noise. For example, individual data lines not corresponding to expected signal patterns may be eliminated or modified. The multivariate data may be constrained to eliminate multivariate combinations or regions not generated by physiologic signal. The constraints may be based on a model, experimental, a priori data, a standard 12-lead ECG from that patient, a standard set of constraints from experience, or data obtained inside and/or outside the interfering environment (e.g., with and without the static magnetic field, and/or the gradient switching). The residual multivariate data may be fit to a parametric model that includes a representation of the important features, the multivariate data may be analyzed statistically for correlation with specific features, or a neural network may be applied to extract the desired features.

A synthetic signal is then generated showing the desired features more clearly, optionally corresponding to specific standard lead combinations, and conforming to simple rules such as: R-wave is highest peak. Alternatively, the R-wave may represent the expected height for a specific ECG lead combination, but with a superimposed spike, analogous to a pacemaker spike, so that the highest net peak coincides temporally with electrical activation of the large chambers. In addition, small spikes may be added to the out-going signal following the standard presentation, to represent the numeric value of ST segment deviation, e.g., two and a half up-going spikes after the T wave to indicate 2.5 mm ST elevation.

The present invention together with the above and other advantages may best be understood from the following detailed description of the embodiments of the invention illustrated in the drawings, wherein:

### BRIEF DESCRIPTION OF THE DRAWINGS

FIG. 1 is a block diagram of the multivariate characterization monitoring system according to principles of the invention;

FIG. 2 shows details of the sensor system;

FIG. 3 shows details of the first processing unit;

FIG. 4 is a block diagram of the management of the multivariate data;

FIG. 5 is a diagram of the data processing logic;

FIGS. 6A-6J show examples of signals from standard ECG's;

FIG. 7 shows examples of output from the present invention; and

FIG. 8 demonstrates how multivariate signal characterization enables feature extraction where individual variables fail.

### DETAILED DESCRIPTION OF PREFERRED EMBODIMENTS

A preferred embodiment of the present invention incorporates therein a monitoring system that uses multiple electrodes to create a multivariate characterization of the status of the heart. This system derives, from the multivariate data, a synthetic or composite ECG for specific purposes. A synthetic ECG is one regenerated or constructed by computer, in this case based on specific information extracted from the multivariate data. The present invention analyzes data from multiple leads to generate a multivariate characterization of the events of interest. That data is used to identify specific features such as timing of the electrical activation of the large chambers, to specify the R-wave in the computed or simulated signal output.

In the preferred embodiment, wire from an electrode that makes contact with the chest wall is paired and twisted with wire from the same local area on the chest wall but not making electrical contact with the chest. The wires from these paired locations are resistive to reduce pick-up of stray signals, e.g., 60 cm carbonized wires with 200,000 ohms impedance end to end. A plurality of such lead pairs are applied to the anterior and/or posterior and/or side(s) of the chest wall as an array, harness, vest, partial vest or shoulder holster. The vest looks like a lightweight elasticized garment with skin electrode contacts distributed to make electrical contact at locations distributed over the chest. The number of contact points is at least two, and may be distributed to include chest wall anterior and/or posterior and/or lateral to the heart.

The wire leads go to a battery-powered magnetic field-compatible processing unit (both the wire leads that contact the skin, and the wire leads that optionally are paired with contacting leads but do not make skin contact). The leads that do not make skin contact provide signals not related to the ECG, so that such signals, when found also on the skin-connected leads, can be eliminated. This operation is called common mode rejection, or CMR. All processing may be completed in that unit, or the signals may be multiplexed and converted to optical or other signal for transmission to a second processing unit.

The processing compares multivariate signals to training data to identify desired features of the signal, e.g. electrical activation of the smallest chambers (P-wave), electrical activation of the large chambers (R-wave), early repolarization (ST-segment), peak repolarization (T-wave), respiratory phase from baseline artifact, and wave morphologies. Likewise the processing to compare with training data and/or measured reference data can be used to identify undesirable features such as aortic pulsation and gradient switching artifacts. Training data comprise multivariate signals acquired for this invention, empiric data, standard signals acquired from standard positions, on the same patient as a preliminary evaluation, on the same patient by scanning in a prior standard ECG, and/or on different or made up subjects. Training data may also include data collected on gradient effects and magnet effects. The training data represent the features of interest, expected ranges of values and covariance as a function of time, and expected signal disturbances.

A synthetic signal is produced from the identified features showing the desired features more clearly and optionally conforming to simple rules that promote clarity such as: R-wave is highest peak, baseline is flat, P-wave is distinct, ST-segment deviation if present is clear and measurable in millivolts (or millimeters corresponding to voltage) deviation from the flat baseline. Optionally, a sequence of voltage spikes following the T wave will count how many millimeters or tenths of millivolts of ST segment deviation (using half-height for half a millimeter). Thus the synthetic signal is a signal generated by computer containing key features of interest such as P-wave, QRS, ST-segment deviation, T-wave, in a clean form. It may represent any selected view such as any of the standard 12 lead combinations or extended alternate views that may show maximal R wave or maximal ST segment deviation. The synthetic signal allows presentation of “in-between” or interpolated views that correspond better to conventional standards than the possibly non-standard positions observed.

The heart generates current distributions, from its movement of ions, resulting in voltages on the chest that are basically a continuous function of position sampled. We determined that the signal that would have been observed at an un-sampled position may be estimated accurately from the signals at neighboring positions; the correspondence between multivariate observations and standard lead position data enable prediction of the standard views from the multivariate observations, e.g., by curve fitting.

The computed results may be expressed as a synthetic analog ECG signal. Also, the predicted signal need not be constructed directly as voltages vs. time. Alternatively, it may be constructed from basis elements reflective of the desired information content: timing of the P wave deflection, interval between P and QRS, timing of the R wave deflection, severity of ST segment displacement, presence or absence of T wave inversion. Such information elements suffice to generate a simulation signal that accurately reflects those variables based on the multivariate data, but presents them as a clean, very easily understood standardized view, free of noise and artifacts.

The user may elect to preserve R wave height and/or form in the simulated ECG. Then, rather than making the R-wave the maximal peak by design, a narrow upward spike may be superimposed, similar to the signal of a pacemaker, so that legacy R-wave detectors will unfailingly follow the timing of the electrical activation of the large chambers of the heart. In addition to visually communicating specific information, the simulated ECG provides a standard input to pass the accurate interval tracking to legacy systems such as threshold R-wave trackers on MRI systems.

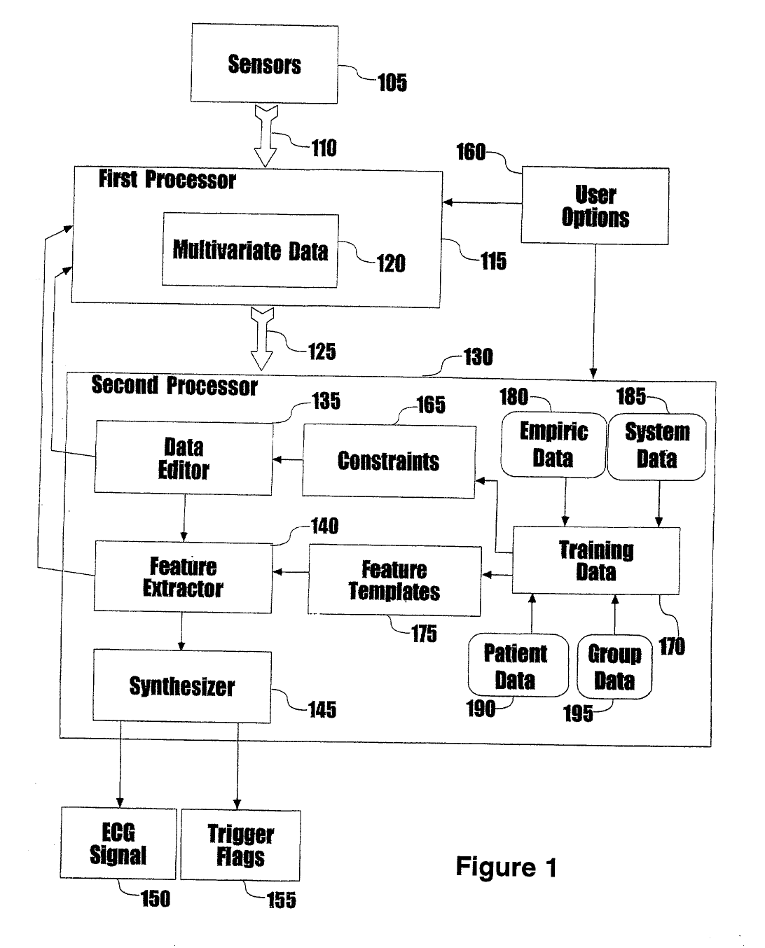

The major components of the present invention are shown in FIG. 1. First, a plurality of sensors (105) detect physiologic signals. Those signals are linked by linkage (110) to a first processor (115). The first processor (115) converts the signals to multivariate data (120). The multivariate data (120) from the first processor (115) may be linked by linkage (125) to a second processor (130). The second processor (130) applies a data editor (135), a feature extractor (140), and an output synthesizer (145) to the multivariate data (120), to create signal output (150), and/or trigger flags (155) for triggering or gating and/or accounting for rhythm changes. The first processor (115) may receive control input from user options (160), from the data editor (135), and from the feature extractor (140). The data editor (135) may receive control input from constraints (165), which may receive input from training data (170). The feature extractor (140) may receive input from the feature templates (175), which may receive data from the training data (170). The training data (170) may receive data from user options (160), empiric data (180; data that serves as a model of co-variant ranges, patterns, and parameters), system data (185; data from equipment such as MRI indicating what signals or noise the equipment may generate or induce), patient data (190; data from the patient indicating target signal co-variant ranges, patterns, and parameters), and group data (195; data from a group or population indicating expected co-variant ranges, patterns, and parameters). The output synthesizer (145) may receive control input from user options (160).

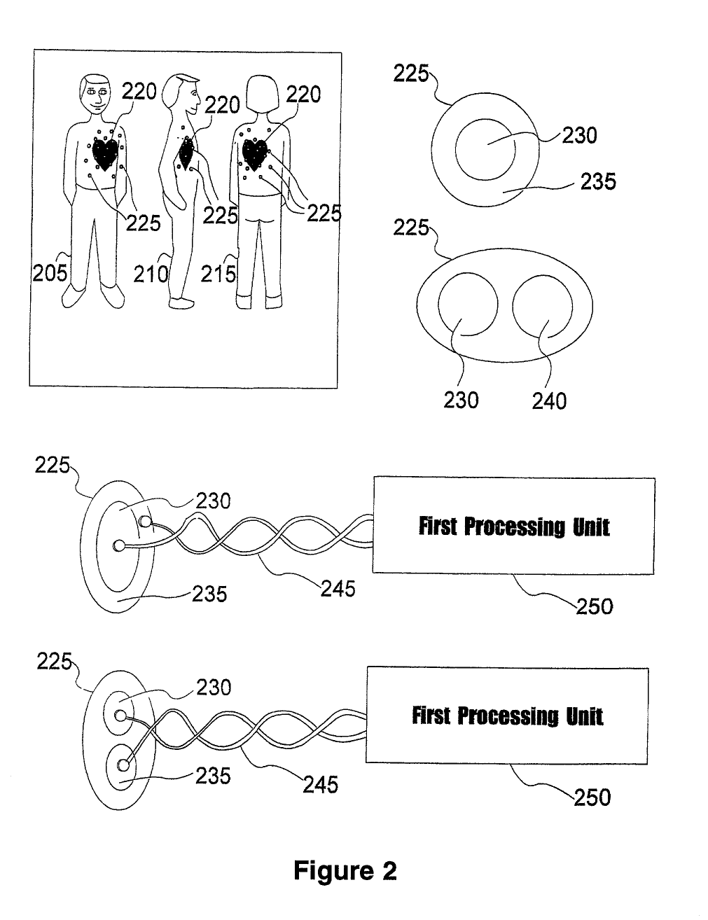

Details of the sensor system are shown in FIG. 2. A patient (anterior view 205, posterior view 210, lateral view 215) has a plurality of sensors applied in contact with the skin surface. The present invention provides great latitude as to the number, distribution and arrangement of the sensors on the skin, with no requirement for orthogonality, regular spacing, or alignment in rows or columns. The preferred distribution of contact points includes anterior (205), posterior (210) and lateral (215) contacts on the thorax, above, at, and below the general level of the heart (220).

Each sensor (220) has a conducting contact (225, 230) that makes electric contact with the skin. The contact may be maintained, for example, by adhesive, one or more elastic straps, or an external vestment. Each conducting contact (230) optionally has an associated non-conducting ring (235) or a closely associated non-conducting contact (240), referred to as a null terminal.

The plurality of sensors (230) and any associated null terminal contacts (235 or 240) may be interconnected by a non-conducting material, e.g., a vestment, to preserve their relative or absolute positions, and avoid tangling.

Each sensor (230) and any associated null terminal (235 or 240) links by a linkage (245) to the first processing unit (250). The linkage (245) of a paired sensor (230) and null terminal (235 or 240) may be twisted pair wire so they have similar pick up of any stray signals. The linkage may consist of carbonized wire for high impedance, e.g. 200,000 ohms, to minimize pick up of stray signals. All components in FIG. 200 may be non-magnetic.

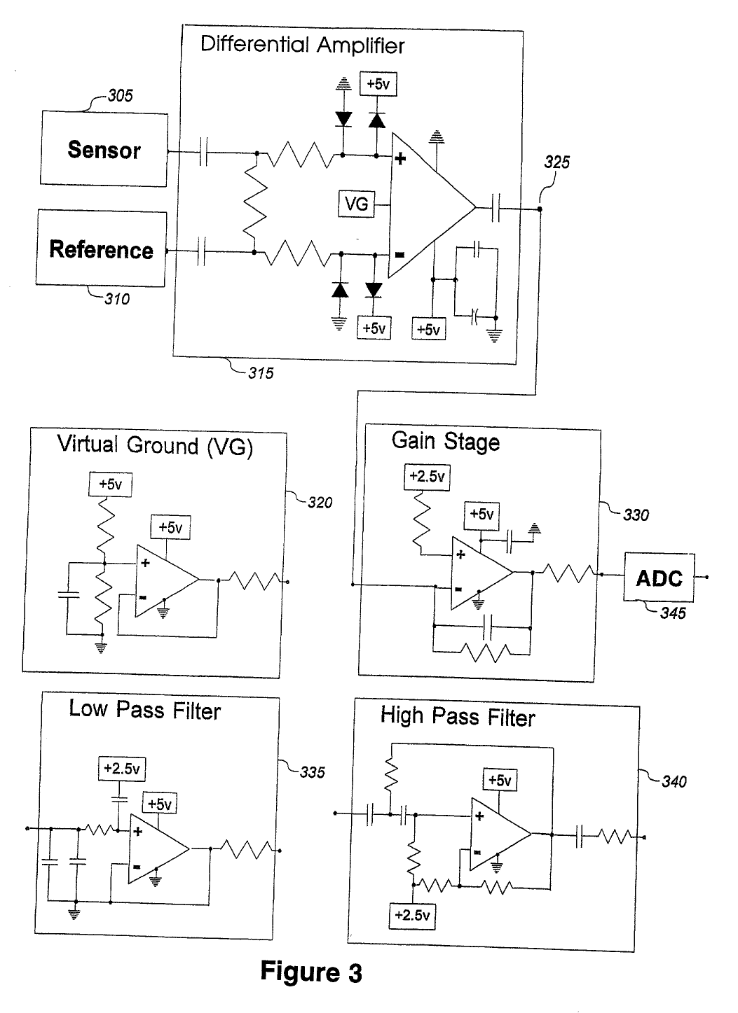

Details of the first processing unit are shown in FIG. 3. Linkages from a sensor (305) and a reference (310) connect to a differential amplifier (315), or an instrumentation amplifier, for common mode rejection of unwanted or stray signals present in both linkages. The reference (310) can be a said null terminator, or a single common sensor serving as reference, or a member of a set of sensors. For example, all possible pairings of sensors may be used. The center terminal of the differential amplifier may be linked to the output from a virtual ground generator VG (320), to enable DC bias to place the incoming voltages in an appropriate range for the differential amplifier (315). The output (325) from the differential amplifier may go to a gain stage (330) to prepare the signal for analog to digital conversion. Optionally, a low pass filter (335), and/or a high pass filter (340) may be placed before and/or after the gain amplifier (330), to constrain the signal to frequencies of interest. Optionally, the DC offset from the virtual ground generator VG (320), and/or the amount of amplification in the gain stage (330), and/or the pass levels for the filters (335, 340), may be set by remote linkage from a second or third processor or from user input. The signal next undergoes analog to digital conversion.

The analog-to-digital conversion may be accomplished by an analog to digital converter or ADC (345), which may be a stand alone component or integrated with a microprocessor, e.g., Microchip PIC16C73B, or preferably with sigma-delta encoding and 15 bit resolution. The ADC converts the set of signals from the sensors to digitized multivariate data.

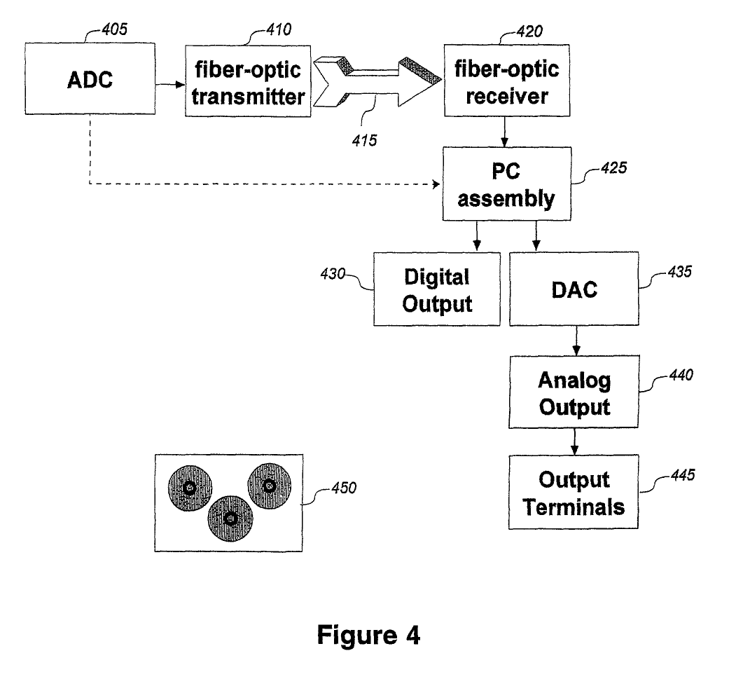

Management of the digitized multivariate data from the ADC is shown in FIG. 4. Although not required for the main objectives of the present invention, the preferred embodiment links the digitized multivariate data from the ADC (405) are linked to a fiber-optic transmitter (410), transmitting the multivariate data over a fiber-optic linkage (415) to a fiber-optic receiver (420). The fiber-optic transmitter can use a light emitting diode or a dedicated encoder, e.g., applying 890 nanometer short wavelength light to support up to 125 megabits/second data transmission. The fiber-optic linkage (415) avoids further pick-up of stray signal, and allows further processing to be placed remote from interfering equipment such as an imaging system or strong magnetic fields.

The fiber-optic receiver (420), or the ADC (405) directly, is linked to a PC assembly (425) for analysis of the multivariate data features and synthesis of output. The output from the PC assembly (425) may be used in digital form, with digital outputs (430) for ECG and/or respiratory triggering or gating or other condition flags. In particular, one condition flag may indicate end-expiration, and thus return of the diaphragm, and the heart riding on the diaphragm, to a standard position. Another condition flag may report whether the preceding R-R interval was within tolerance of the mean R-R interval for that patient. That serves to indicate that the filling time from the preceding interval is standard, and so the heart volume at the current trigger is standard for that patient (thus providing a mechanism for reliable triggering or gating even in the presence of marked rhythm disturbances such as atrial fibrillation). Also, the output from the PC assembly may be linked to a digital-to-analog converter (435), producing analog output signal (440). The analog output signal (440) may be linked to output terminals 445 similar to those on standard electrodes, so that an imaging system requiring ECG signal may interface to these output terminals (445) as if they were standard electrode snap connectors. The terminal electrodes are shown from top view (450), and one in side view (455).

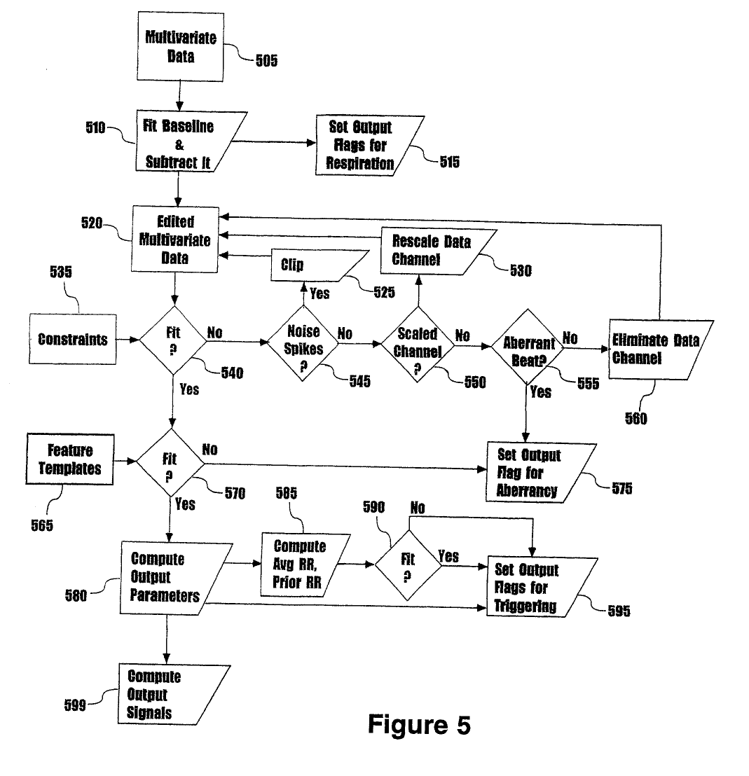

Logic operations and data flow for an embodiment of the data processing are shown in FIG. 5. Multivariate data (505) is analyzed by low frequency curve fit with median filter to fit the respiratory baseline artifact and subtract it (510). The results of the baseline fit are used to set output flags for respiration status (515). The flags are cleared when read, and reset by further data according to the present status. The baseline subtraction results in edited multivariate data (520). The edited multivariate data (520) also results from clipping spikes (525; if data deviate from expectation momentarily, e.g., apply median filter), resealing (530; if data agree in form but differ in amplitude), and data channel elimination (560; if the data from a sensor is unreliable). Based on the edited multivariate data (520) and constraints (535; which reflect expected temporal evolution of the multivariate data), a comparison (540) determines if the edited multivariate data (520) fit the constraints (535). If they do not fit (540), the data is examined further for noise spikes (545), scale change (550), aberrant beat or ectopy (555), or unreliable data channels (560). If the edited multivariate data (520) does fit (540) the constraints (535), then feature templates (565) are fit (570) to the data. If the shape and/or timing parameters do not fit well, the data may yet be flagged as aberrant (575). If the fit (570) is good, then output parameters (580) are computed. These parameters describe the timing and/or shape of important signal components (QRS, RR-interval, ST-segment deviation, etc.). From the output parameters (580), average RR interval, standard deviation, and last RR interval (585) are computed. The last RR interval is compared to the statistical summary (590) to determine if the filling time offers a standard anatomic filling for imaging, and triggering flags (595) are set accordingly. The timing and/or predicted timing of the R wave activation also sets triggering flags (595). In addition to setting flags, the computed output parameters 580 are applied to synthesize output signal (599). The output signal (599) reports a clean ECG signal in any desired view with a spike superimposed to mark the R wave trigger, with ST segment deviations corrected for baseline artifact, machine effects (via constraints, which are built from information about the patient, expected signals, gradient effects, and magnet effects), and noise. Optionally, the ST-segment deviations and/or other features may represent a running average over a user-selected time period. Also, a series of spikes may be added after the T wave to count out the amount of ST segment deviation as described.

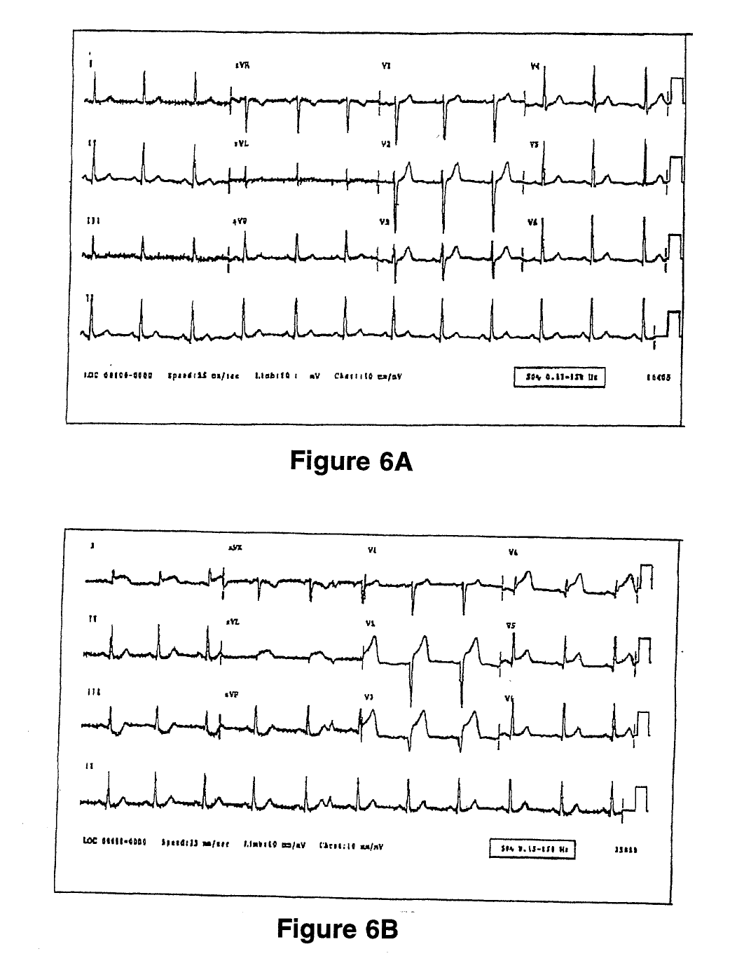

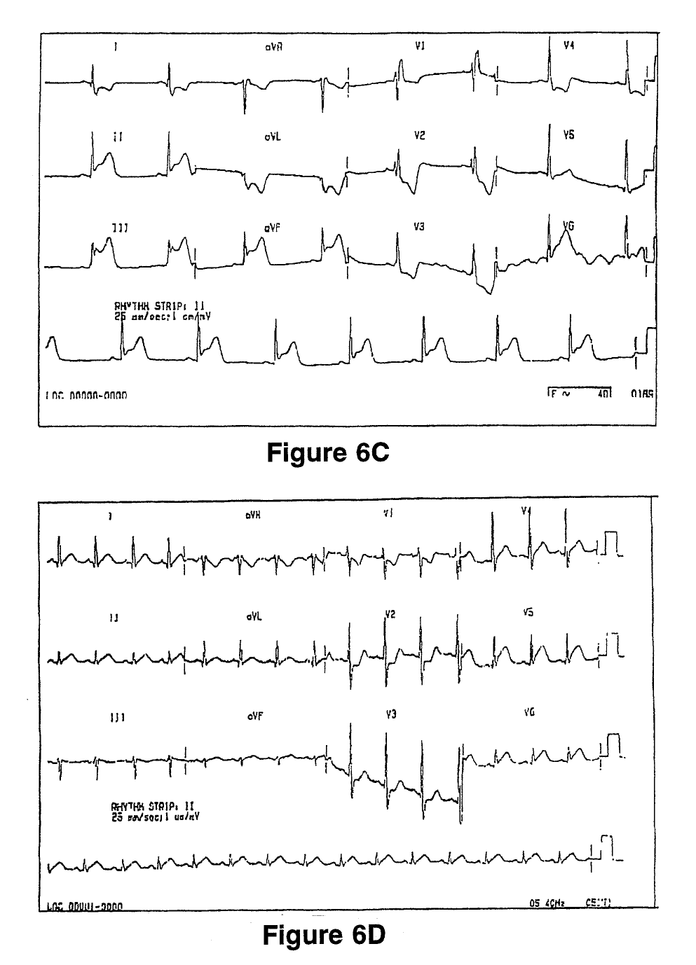

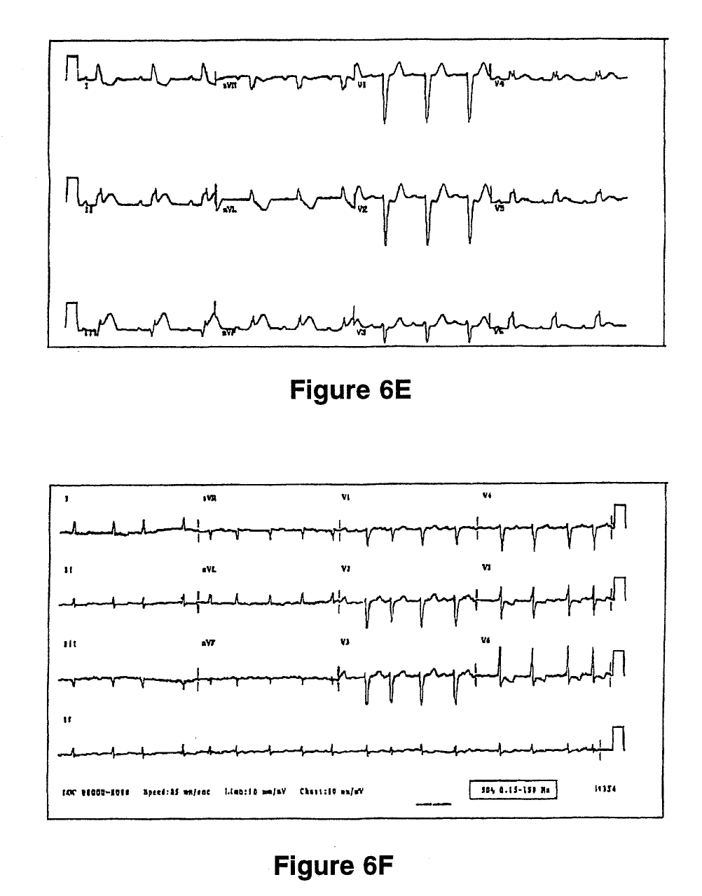

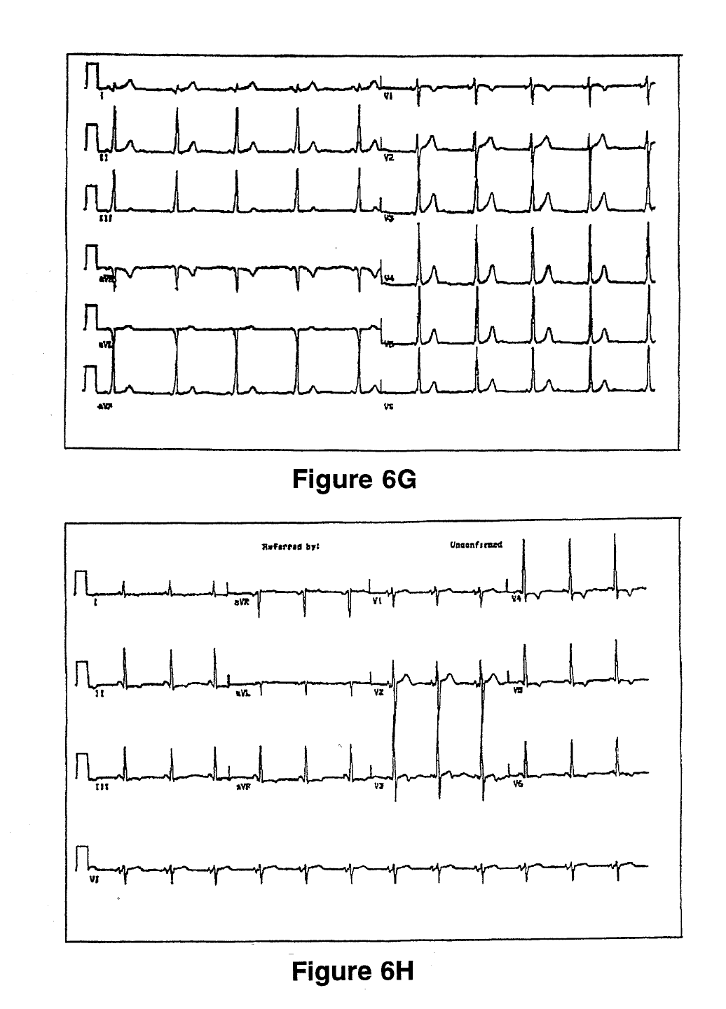

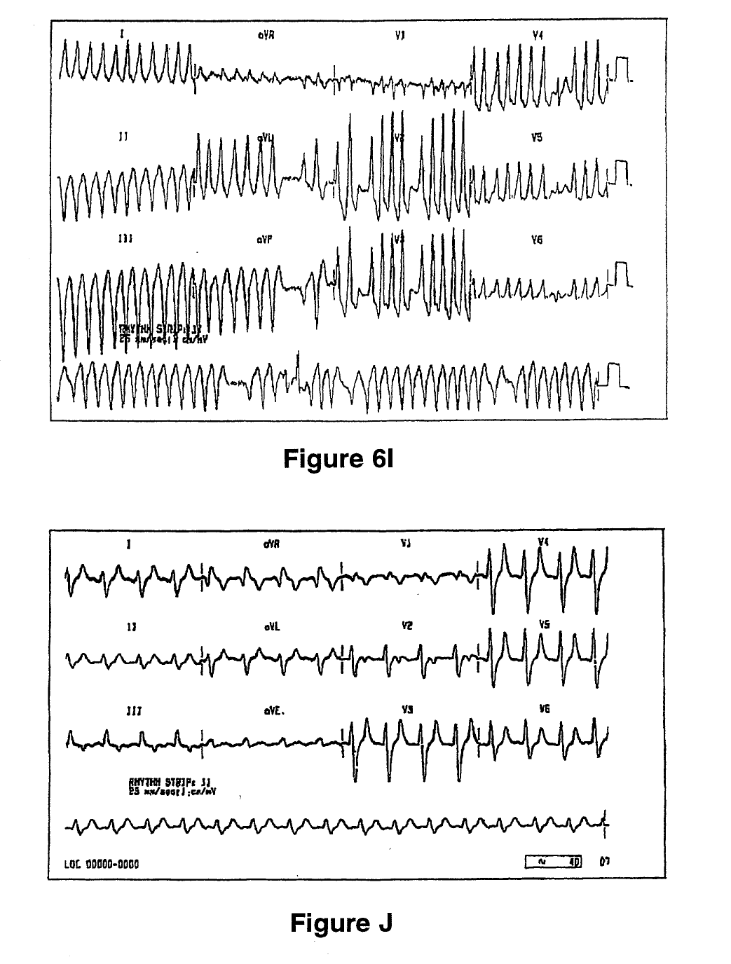

A normal 12 lead ECG is shown in FIGS. 6A. The data is organized to show several beats from each lead, as labeled, plus a longer “rhythm strip” from lead II as the bottom row. Note the noise in leads I, III, aVL, all relating to low quality signal from the left arm contact. Multivariate evaluation would provide alternatives, so the noisy data line could be circumvented. Each P wave is followed by a QRS. The shape of the P wave is normal for the subject; in lead II the height is less than 2.5 mm, and the width is less that 0.11 seconds. The rate is between 60 and 100/minute with less than 10% variation. The P-R interval (beginning of P to beginning of R) is steady and between 0.12 and 0.20 seconds. The QRS heights are positive in leads I and avF, indicating a normal “axis” or principle frontal direction of activation, and nowhere are they high enough to indicate heart enlargement. The width of the QRS is less than 0.12 seconds. The shape is normal for the subject; no significant Q waves, no extra components. The QT interval (beginning of QRS to end of T wave), adjusted for the rate by dividing QT by the square-root of the preceding RR, is 0.42 seconds. The ST segment is not elevated or depressed over the baseline extrapolated from the PR segment. The shape of the T wave is normal for the subject; not too tall, not generally flat or inverted, and generally in the same direction as the QRS.

Examples of ECG's are shown in FIGS. 6A-6J. FIG. 6B shows a pattern or ST segment shifts which indicate new infarction, or cell death, in the anterior wall of the heart. (Patient with anterior wall myocardial infarction) The ST segment elevation is most prominent in leads v2 and v3. Notice also the loss of R wave heights compared to the normal ECG of FIG. 6A. That loss of R wave makes it more difficult to gate or trigger imaging by standard methods. In current practice, the imaging technician can spend half an hour or more, trying different lead placements and combinations, seeking a tall R wave for triggering. Note ST elevations in v leads particularly v2, v3, the loss of R waves v2, v3 and the reduced R wave in v4. Standard image gating from any of the standard chest leads v1-v3 would fail to detect the R wave.

An ST elevation pattern shown in FIG. 6C occurs with inferior wall infarction. (Patient with inferior wall myocardial infarction) Note the ST segment elevations in leads II, III, and aVF. There are reciprocal changes in the ST and T waves of the v leads, but it is vital to examine the former leads or equivalent views to recognize the life-threatening condition. Also, there is a change in the morphology or form of the QRS: a second peak, or R′, due to damage in the electrical conduction system in the heart. Such changes commonly interfere with standard ECG trigger methods, as the second peak may trigger instead of the first. Note ST elevations in leads II, III, and aVF, with reciprocal changes in I, a VL, v2-v4. Also note the change in the QRS of v1 and v2 to RSR′, evidence of right bundle branch block (abnormality in the electrical activation pathways). It is very important to recognize evidence of acute injury and/or ischemia.

Abnormal Q waves in the posterolateral wall of the heart shown in FIG. 6D indicate an older infarction. (Patient with posterolateral wall myocardial infarction) Note the changes in R wave heights, which could interfere with standard triggering, especially after placement in a magnet (not shown) when the T wave is effectively much taller due to signal from blood in the great vessels moving in a strong magnetic field. Note Q waves in I, aVL, v6 and decreased R wave in II, III, F, v5, v6. Note also baseline drop in v3 which is not helpful in the analysis, and is fully corrected in the present invention.

A conduction abnormality as shown in FIG. 6E, not uncommon in patients with heart disease, contradicts the assumption of filtered EKG methods for EKG triggering, that the R wave is narrow. (Patient with left bundle branch block) The pattern shown in FIG. 6E indicates left bundle branch block, resulting in a change in width, height, and form of the QRS. Note the substantive loss of R wave height. With standard systems, it may prove impossible to gate or trigger. Note the wide QRS, and severely reduced R wave in most leads. The usual image gating from any of the standard chest leads v1-v4 would fail to detect the R wave.

Irregular rhythm due to atrial fibrillation as shown in FIG. 6F also interferes with standard triggering. Standard triggering fails here because the preceding RR interval has very variable length; the amount of time that the ventricle fills with blood varies, the heart size and position varies. (Atrial Fibrillation and Digoxin Effect) The present invention tracks the preceding and average RR interval, so that an imaging system can reject data with long or short filling times, enabling high quality imaging in spite of the arrhythmia. Also, due to variable times of recovery due to varied RR intervals, the R wave may widen as in left bundle branch block. Note the irregular rhythm. Standard image triggering works poorly here because a proper R wave trigger corresponds to variable filling times, and thus different sizes and positions of the heart. Also note changes in the ST segments and T waves related to the medication.

A conduction abnormality from the small chambers to the large chambers as shown in FIG. 6G can result in a short PR interval, and a change in the shape of the QRS: Wolf-Parkinson-White conduction. (Patient with Wolf-Parkinson-White conduction abnormality) This is a congenital condition. The change in shape of the QRS could interfere with systems that rely on narrowness of the R wave as part of the trigger. Note the short interval between the P waves and the R waves and the slurred initiation of the R wave followed by an R wave peak at the normal P-R interval.

Another conduction abnormality from the small chambers to the large chambers as shown in FIG. 6H that does not change the shape of the QRS is called Lown-Ganong-Levine conduction. (Patient with Lown-Ganong-Levine conduction abnormality) It is helpful to recognize such abnormalities because they are associated with rhythm disturbances, especially under stress. Note the short interval between the P waves and the R waves, without a slurred upstroke.

A common rhythm disturbance that may occur with conduction abnormality from the small chambers to the large chambers as shown in FIG. 6I looks similar to a deadly emergency: atrial fibrillation plus Wolf-Parkinson-White conduction. (Patient with Wolf-Parkinson-White syndrome and atrial fibrillation) If the doctors did not note the abnormality before the rhythm change, they might well think this is ventricular tachycardia, a very different potentially life-threatening condition treated by applying a strong electric shock. In standard imaging systems, this might be confused also with gradient switching artifact. The present invention substantively eliminates gradient switching signal, avoiding that potential confusion. Note the rapid irregular timing, esp. in the rhythm strip in the bottom row, and the wide QRS due to the conduction abnormality.

Elevation of potassium level results in tall peaked T waves as shown in FIG. 6J. In standard imaging systems, this could be easily missed, because tall peaked T waves are seen inside magnets routinely, due to signal produced from pulsation of blood in large vessels in the strong magnetic field. High potassium levels can be life threatening. The present invention distinguishes and substantively removes the signal from the magnet, avoiding that potential confusion. Note the tall peaked T waves, which are taller than the R waves in the chest leads v3, v4, I, II, and aVL. A standard image triggering system would trigger off the wrong wave in such circumstance.

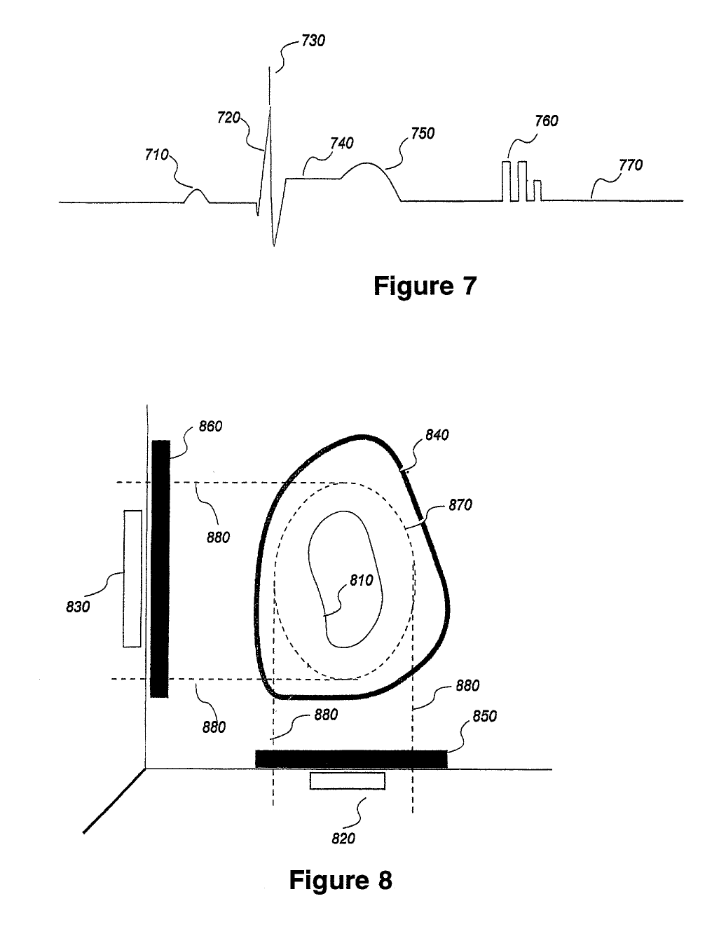

An example of synthetic signal from the present invention is illustrated in FIG. 7, which shows a clear P wave (710), QRS (720), pace spike (730), ST segment (740), T wave (750), markers measuring the 2.5 millimeter ST segment elevation (760) and a perfectly flat baseline (770). The synthetic ECG can produce all the standard views, and the extra views that are sometimes important (V7, V8, V9, V4R, and V5R, etc.). Unlike the actual ECG's, the features are even easier to evaluate, because of the flat baseline, substantive elimination of noise, and clear definition of the components. The pace spike (730) does not represent a pacemaker, but rather is a superimposed signal that will trigger legacy imaging systems that simply look for the tallest wave. Optionally, the pace spike will be suppressed by short or long preceding RR intervals, or a separate flag will indicate the occurrence of short or long preceding RR intervals, for effective gating in spite of changes of rhythm. The markers (760) facilitate recognition of significant changes in ST segment height, which will be very important in one of the newer applications of imaging, assessing blood arrival to the heart and/or wall motion or thickening during stress testing.

A fundamental advantage of the multivariate method of the present invention is illustrated in FIG. 8. For convenience of drawing, the image is on a flat, or two-dimensional region, but is to be understood to represent a region in a plurality of dimensions (distinct coordinates, or data channels), i.e., a multivariate space. The figure shows a region (810) and another region (840). The hollow bars (820, 830) show the projection of region 810 onto the each of two coordinate axes. The solid bars (850, 860) likewise show the projection of region 840 onto each of two coordinate axes.

The projections correspond to the one-dimensional projections of the electrical activation of the heart that are used for standard ECG leads. Recall that the fact that the heart is not simply an electric dipole means that more than 3 dimensions of data may be required to describe well the electrical activation of the heart; even a standard 12 lead system based on 10 electrodes does not suffice for all subjects.

In neither projection (860 vs. 830, 850 vs. 820), nor in this example, any diagonal projection that could be generated from them, are the projections of the regions (810, 840) separable. The projections overlap substantively. That means the corresponding signals cannot be separated, e.g., by filtering, in the projections. However, in the multivariate space, one can define a border (870) that complete separates 810 from 840. The projections of that border (880) still do not separate the projections of the regions. Such a multivariate border can be easily defined by constraints, based on observing the signal positions from various sources in the multivariate space, for example by taking a one-, or two-, standard-deviation border around the multivariate span of the desired signal, in space-time, static, or as a dynamic border changing throughout the cardiac cycle. We have determined that magnetic field gradient-induced signals, and much of the noise and other artifacts, can be completely separable from the desired signal in multivariate space. Consequently, definition of a multivariate border (870) can separate the signals (810, 840) in multivariate space. Application of the constraints eliminates the unwanted signals.

It is to be understood that the above embodiment descriptions are simply illustrative of the principles of the invention. Various and other modifications and changes may be made by those skilled in the art that will embody the principles of the invention and fall within the spirit and scope of the claimed invention.

What is claimed is:

1. A method of monitoring physiologic signals produced by a live body organ comprising the steps of:
(a) positioning a plurality of physiologic signal sensors at different positions in relation to an area of said live body organ for providing multivariant physiologic signals of different but not independent character;
(b) inputting said physiologic signals to a data processor that performs the steps of
(c) converting said physiologic signals to derive desired data for monitoring changes and identifying timing of events relating to said live body organ under examination, said derived data being based upon multidimensional analysis of other data to derive said desired data.

2. The method of claim 1 further comprising the step of generating a voltage spike or flag used to trigger imaging systems.

3. The method of claim 1 wherein said derived new data includes a voltage offset of a segment of a waveform of continuous data, used to identify the presence of or changes in ischemia, expressed as a voltage offset.

4. The method of claim 1 wherein said derived data includes a voltage offset of a segment of a waveform of continuous data, and further comprises the step of identifying the presence or changes in ischemia, expressed as a series of voltage spikes that count out the segment deviation in millimeters or tenths of millivolts.

5. The method of claim 1 wherein said derived data includes representations of a respiratory cycle derived from baseline undulations in EKG signals.

6. The method of claim 1 further comprising the step of using sensors of magnetic gradient switching as reference data to eliminate or isolate their contribution to signals.

7. The method of claim 1 further comprising the step of using data derived from other data and/or variance to define a multivariate volume about desired signals to separate desired from undesired signals.

8. The method of claim 1 further comprising the step of using derived timing triggers to compute and/or forecast comparable filling periods to in turn identify times for comparable positions for image collection or other action, despite the presence of disturbed rhythms.

9. The method of claim 1 wherein said live body organ comprises a heart.

10. Method of performing diagnostic testing of the heart and for enhancing the clarity of a display of features of interest, relating to evaluating the health of a patient's heart under examination, comprising the steps of:
(a) positioning a set of multiple electrical pickup devices in relation to the patient's skin for producing multivariant data of the electrical activation of said heart;
(b) applying said multivariant data to a data processor, which responds to receipt of said multivariant data; and
(c) comparing said multivariant data with training data to derive descriptive values that are applied to template components, and generating synthetic composite ECG electrographic data in an easily understood view, indicating various heart conditions where such heart conditions may include the nature and/or timing of P waves, QRS waves, ST segment deviation, T waves, and/or respiratory motion.

11. The method of claim 10 further comprising the step of substantially reducing an artifact produced by aortic pulsations that can interfere with clear readings of said synthetic composite ECG electrographic data.

12. The method of claim 11 further comprising the step of analyzing said multivariant data by a low frequency curve fit or filter to extract the respiratory baseline artifact and subtract it to produce a flattened baseline.

13. The method of claim 10 further comprising the step of superimposing upward trigger spikes upon R wave heights for ensuring that legacy R-wave detectors for following timing of the electrical activation of large chambers of the heart.

14. The method of claims 11 further comprising the step of superimposing upward trigger spikes upon R wave heights for ensuring that legacy R-wave detectors for following timing of the electrical activation of large chambers of the heart.

15. The method of claim 12 further comprising the step of superimposing upward trigger spikes upon R wave heights for ensuring that legacy R-wave detectors for following timing of the electrical activation of large chambers of the heart.

16. The method of claim 10 further comprising the steps of:
determining if said multivariate data fits constraints which define limits of acceptable variations relating to one or more of the following: noise spikes, scaled channel, aberrant heart beats or otherwise unreliable data; and
editing said multivariate data to cause said multivariate data to fit the constraints if said multivariate data does not fit.

17. The method of claim 11 further comprising the steps of:
determining if said multivariate data fits constraints which define limits of acceptable variations relating to one or more of the following: noise spikes, scaled channel, aberrant heart beats or otherwise unreliable data; and editing said multivariate data to cause the data to fit the constraints if said multivariate data does not fit.

18. The method of claim 12 further comprising the steps of:
determining if said multivariate data fits constraints which define limits of acceptable variations relating to one or more of the following: noise spikes, scaled channel, aberrant heart beats or otherwise unreliable data; and editing said multivariate data to cause the data to fit the constraints if feasible if said multivariate data does not fit.

19. The method of claim 10 further comprising the steps of:
comparing said multivariate data to training data to identify desired features for display which can include production of P-wave, R-wave, ST-segment, T-wave, respiratory phase from baseline artifact, and wave morphologies, and wherein said training data can represent the features of interest, expected ranges of values and covariance as a function of time, and expected signal disturbances; and
displaying said composite ECG electrographic data in response to a favorable comparison.

20. The method of claim 11 further comprising the steps of:
comparing said multivariate data to training data to identify desired features for display which can include production of P-wave, R-wave, ST-segment, T-wave, respiratory phase from baseline artifact, and wave morphologies, and wherein said training data can represent the features of interest, expected ranges of values and covariance as a function of time, and expected signal disturbances; and
displaying said composite ECG electrographic data in response to a favorable comparison.

21. The method of claim 12 further comprising the steps of:
comparing said multivariate data to training data to identify desired features for display which can include production of P-wave, R-wave, ST-segment, T-wave, respiratory phase from baseline artifact, and wave morphologies, and wherein said training data can represent the features of interest, expected ranges of values and covariance as a function of time, and expected signal disturbances; and
displaying said composite ECG electrographic data in response to a favorable comparison.

22. The method of claim 16 further comprising the steps of:
comparing said multivariate data to training data to identify desired features for display which can include production of P-wave, R-wave, ST-segment, T-wave, respiratory phase from baseline artifact, and wave morphologies, and wherein said training data can represent the features of interest, expected ranges of values and covariance as a function of time, and expected signal disturbances; and
displaying said composite ECG electrographic data in response to a favorable comparison.

23. Apparatus for monitoring physiologic signals produced by a live body organ, comprising:
(a) a plurality of physiologic signal sensors, said sensors being placed at different positions in relation to said live body organ to provide multivariant physiologic signals of different but not independent character;
(b) data processing means coupled to said signal sensors for converting said physiologic signals to derive a composite set of desired data more reliable for purposes of monitoring changes and identifying timing of events relating to said live body organ under examination, derivation of said desired data being based upon multidimensional modeling of observed data in comparison to training data

24. The apparatus of claim 23 wherein said coupling comprises a plurality of pairs of twisted electrically conductive leads, each pair being associated with a corresponding sensor, and a first lead of each pair being in electrical contact with a particular signal sensor and a second lead of each pair being electrically disconnected from said particular signal sensor but terminated adjacent thereto.

25. The apparatus of claim 23 wherein each sensor makes electrical contact with a skin portion of a patient under examination.

26. The apparatus of claim 24 wherein each sensor makes electrical contact with a skin portion of a patient under examination.

27. The apparatus of claim 23 wherein said desired data is generated by applying derived descriptive values to template elements.
